(19) United States
(12) Patent Application Publication
(10) Pub. No.: US 2024/0225443 A9
(43) Pub. Date: Jul. 11, 2024

(54) SYSTEM AND METHOD TO OBTAIN INTRAOCULAR PRESSURE MEASUREMENTS AND OTHER OCULAR PARAMETERS

(71) Applicant: AeyeDx, Inc., Miami, FL (US)

(72) Inventors: Alberto O. GONZALEZ GARCIA, Randolph, NJ (US); Freddy S. MORGENSTERN, Wayne, PA (US)

(21) Appl. No.: 18/547,176

(22) PCT Filed: Mar. 4, 2022
(86) PCT No.: PCT/US22/18854

§ 371 Date: Aug. 21, 2023

Related U.S. Application Data
(60) Provisional application No. 63/157,028, filed on Mar. 5, 2021.

(51) Int. Cl. A61B 3/16; A61B 3/00; A61B 3/113; A61B 3/14; G06T 7/00

(52) U.S. Cl. CPC A61B 3/165; A61B 3/0025; A61B 3/113; A61B 3/145; G06T 7/0012; A61B 2560/0431; G06T 2207/10004; G06T 2207/20081; G06T 2207/20084; G06T 2207/30041

(57) ABSTRACT

Methods, systems and apparatus including computer programs encoded on a “mobile device” for measuring intraocular pressure among other parameters of an eye using image processing with deep neural networks. One or more physical processors of the computer system are programmed with computer program instructions which, when executed cause the computer system to obtain an image of the eye to be used for intraocular pressure measurement. One or more physical processors of the computer system are programmed with computer program instructions which, when executed cause the computer system to estimate the intraocular pressure of an eye from a picture of that eye.

### FIELD OF THE INVENTION

The foregoing systems, methods and apparatus described herein pertains generally to diagnosis of eye diseases. More specifically, systems, methods and apparatus described herein relate to detecting and measuring intraocular pressure (IOP) of the eye through the use of a computing platform configured with one or more machine learning models.

### DESCRIPTION OF THE RELATED ART

Intraocular pressure (IOP) is the fluid pressure inside the eye. IOP is an important aspect in the evaluation of patients at risk of glaucoma. Tonometry is the method eye care professionals use to determine IOP. Most tonometers are calibrated to measure pressure in millimeters of mercury (mm-Hg). A non-invasive tonometer is described in U.S. Patent Publication 2010/0152565 A1, herein incorporated by reference as if presented in its entirety. U.S. Patent Publication 2010/0152565 describes the use of tonometry devices without the need of a medical expert. However, the 2010/0152565 publication and similar disclosures, still require the use of a specialized tonometry device even though they are designed for self-measurement. Furthermore, the device described in the 2010/0152565 publication also requires stabilization, maintenance and calibration. For example, the 2010/0152565 publication states that stabilizing the tonometer is required for improved accuracy of the results. If the device is not calibrated or stabilized correctly, then the device produces inaccurate measurements. Similarly, correct usage of the tonometer requires the user to place the tonometer correctly. Devices like the tonometry device of the 2010/0152565 publication can present inaccurate measurements to a user if not used properly. Where the user is not skilled in tonometry usage, the user would not appreciate that the measurements are unreliable or inaccurate. This raises the hurdle for the use of the device by a non-expert person.

Another instrument, by A. O. Reichert of Depaw, NY, utilizes an air applanation technique, which does not require the instrument to touch the eye. Such non-contact tonometry devices can be preferred by the eye care community because it's more comfortable for the patient and easier to administer by the health provider. While some of the above instruments provide reliable estimates of intraocular pressure, they lack portability, reliability or they need expert knowledge. None of the instruments provide accuracy, reliability, portability and accessibility all together for the non-expert user.

Thus, what is needed in the art are apparatus, systems and methods that allow for reliable estimates of intraocular pressure, without the need for expert analysis, while also maintaining portability, accessibility, and reliability.

### SUMMARY OF THE INVENTION

It is one object of the present invention to provide a system to measure intraocular pressure (IOP) that can easily be used outside the health professional's office. The method of the present invention does not require a special tonometer device or a health professional to be present. In one particular implementation, the IOP measurements can be obtained using measurement can be done using a mobile computing device, such as a smartphone or tablet computer. Specifically, the use of non-custom hardware and software platforms allows for the accuracy of measurements with the disclosed system to not be dependent on the techniques or the expertise of the operator.

The IOP measurement method of the present invention is appropriate for use in non-clinical environments and does not require a preparation. However, the measurement method of the present invention can also be used in clinical environments as well. The invention claims should not be construed as limited to self-measurement. It is another object of the present invention to eliminate the mistakes of the end user while taking the picture of the eye. To ensure that another aspect of the present invention provides a neural-net backed eye-detection and camera adjustment module.

Another aspect of the present invention provides an end-to-end system for the self-measurement of intraocular pressure. The method includes the steps of: starting the app on a mobile computing platform configured with one or more image capture deices; directing the image capture device of the mobile computing device of to capture an image of the eye of a user; detecting the eye area and optimizing camera zoom level, focus, light to obtain an image of sufficient resolution of the eye to enable determination of intraocular pressure thereof; preprocessing the eye image for further de-noising and normalization. The mobile computing device is further configured to send image captured of the eye to a neural-net backed IOP classifier. In one or more further implementations, the IOP classifier is configured to receive image data as an input and correlate image data to corresponding IOP output value and other statistical data. In one or more further implementations, the IOP output values are used to generate an output dataset that contains at least the output of the IOP. In one or more further implementations, the generated report is displayed on one or more display devices for review by the user.

In one or more further implementations of the approaches provided herein, a computer system is provided where the computing system is further configured to obtain an image of the eye, or a portion thereof, and evaluate the image according to one or more diagnostic or evaluation machine learning models or modules. For instance, a computer is configured to provide an image captured by an image capture device to one or more neural networks that have been trained to classify or determine the depth of the anterior chamber of the eye of a subject. In an alternative implementation, the computer is configured to provide an image captured by an image capture device to one or more neural networks that have been trained to classify or determine whether the patient is suffering from hypopyon. In yet a further implementation, the neural network is configured to evaluate the content of an image in order to classify or determine whether the patient is suffering from hyphema.

In one or more further implementations of the approaches provided herein, a computer system is provided where the computing system is further configured to obtain an image of the eye, or a portion thereof, and evaluate the image according to one or more inflammation diagnostic or evaluation machine learning models or modules. For instance, a computer is configured to provide an image captured by an image capture device to one or more neural networks that have been trained to classify or determine cellular or other inflammation of the eye of a subject. In another implementation, a computer is configured to provide an image captured by an image capture device to one or more neural networks that have been trained to classify or determine the aperture of the iridocorneal angle of the eye of a subject. In yet a further implementation, a computer is configured to provide an image captured by an image capture device to one or more neural networks that have been trained to classify or determine the thickness of the central corneal area of the eye of a subject.

In one or more further implementations of the approaches provided herein, a computer system is provided where the computing system is further configured to obtain an image of the eye, or a portion thereof, and evaluate the image according to one or more topological diagnostic or evaluation machine learning models or modules. For instance, a computer is configured to provide an image captured by an image capture device to one or more neural networks that have been trained to classify or determine the topology of one or more intraocular structures of the eye of a subject.

### BRIEF DESCRIPTION OF THE DRAWINGS

The invention is illustrated in the figures of the accompanying drawings which are meant to be exemplary and not limiting, in which like references are intended to refer to like or corresponding parts, and in which:

FIG. 1 summarizes the process of smart eye image capturing using an eye-detection neural network.

FIG. 2 lists the layer structure of the eye detection model.

FIG. 3 shows the neural network built using the modules for IOP classification.

FIG. 4 lists the IOP classifier's modules.

FIG. 5 shows the pre-processing steps of training data set, validation data set and the execution data.

FIG. 6 shows the overall system summary using Smart Eye Capturing, Preprocessing and IOP Classifier subsystems.

### DETAILED DESCRIPTION OF PREFERRED EMBODIMENTS OF THE PRESENT INVENTION

By way of overview, the described, system, apparatus, and methods are directed to improved approaches to determining intraocular pressure. Intraocular pressure (IOP) is the fluid pressure inside the eye. IOP is an important aspect in the evaluation of patients at risk of glaucoma. The systems, processes, and apparatus described herein present improvements to the field of IOP measurements. It will be appreciated that IOP values are not a constant, but instead fluctuate throughout the day. Because of this fluctuation, it becomes necessary to measure the pressure of the eye at numerous times of day, including outside of normal health care office hours. The systems, processes, and apparatus described herein utilize one or more commonly available image capture and mobile computing platforms, such as a smart phone or tablet computer to obtain images of the eye of a patient or subject. It will be appreciated that the use of non-custom hardware allows for IPO measurements to be made without the use of complex medical devices. Furthermore, such approaches allow for measurements to be taken by the subject themselves or by an individual that does not have medical training. The systems, processes, and apparatus thus remove the need of expert human to measure the IOP of an eye.

For example, in one non-limiting implementation, a system for measurement of intraocular pressure (IOP) in at least one eye of a subject is disclosed herein. Such a system can include a handheld device; and at least one source of light configured to illuminate the anterior aspect of the eye of a subject. The system also includes at least one at least one imaging sensor configured to capture the light from the anterior aspect of the eye of the subject. The system further includes an optical system configured to convey and focus the reflected and refracted light to the camera sensor. The response signals generated by the camera sensor can be interpreted by one or more processor that are configured by instructions stored in at least one memory storage device. In a particular implementation, the response signals are evaluated by one or more pre-trained neural networks that are trained to correlate response signals to intraocular pressure measurements.

In one or more implementations, the intraocular pressure measurements of the eye of a subject can be made using one or more mobile or portable computing devices. As detailed herein, the intraocular pressure measurement accuracy of measurements made using such a computing or mobile device are not dependent on the technique or the expertise of the operator. Thus, the described systems, methods and computer implemented processes described herein are appropriate for use in non-clinical environments and do not require extensive preparation by the subject. While the devices, methods and systems described herein do not require the intervention of medical experts, such devices described herein can be used in such clinical environments as well. That is, while the presently described systems, methods and apparatus can be used in self-administered testing procedures, the present disclosure is not limited to only to such uses.

In one or more further implementations, the described systems, methods and apparatus are configured to reduce or eliminate user error or other mistakes during IOP measurement processes process described herein. For example, the system, method and computer process described herein also includes one or more neural-net backed eye-detection and camera adjustment modules that process data or information and generate one or more statistical correlations, classifications or categorizations of input data. In yet a further implementation, the one or more apparatus, systems or processes is configured to provide an end-to-end system for the self-measurement of intraocular pressure.

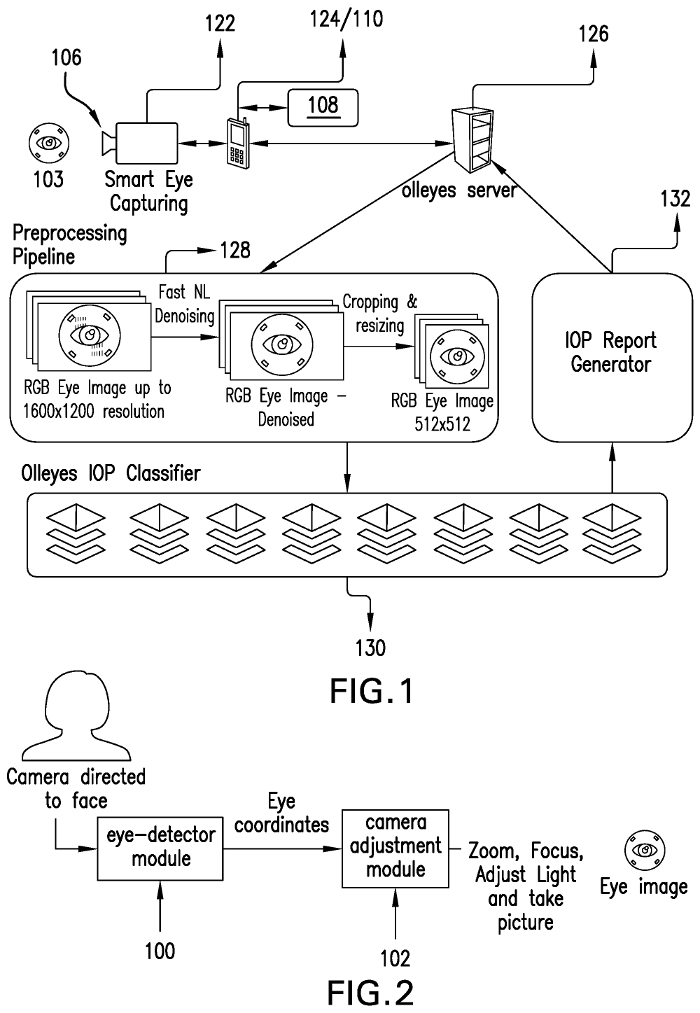

The schematic of an IOP measurement system according to a preferred embodiment is shown in FIG. 1. In one particular implementation, the mobile computing device is depicted as a mobile phone. However, the those possessing an ordinary level of skill in the requisite art will appreciate that the measurement device described herein can be any computing platform that includes or is communicatively coupled to an imaging device. For example, the computing platform can be a mobile computing device, such as a smartphone, camera phone, digital camera, virtual reality devices, such as virtual, augmented and mixed reality goggles, or other such devices.

As shown in FIG. 1, the eye 103 of the subject to be measured is aligned with both the illuminant 106 and an image capture device 122 such that light emitted by the illuminant 106 is reflected off of the eye 103 and received by a sensing element of the image capture device 122. Here, the eye 103 of the subject is an eye under analysis where the intraocular pressure of the eye is desired. As such, any type, manner, or preparation of the eye prior to an intraocular pressure measurement is understood and appreciate.

While one or more implementations include an illuminant 106, it should be appreciated that other configurations described herein do not require an illuminant. In one particular implementation, the illuminant 106 is configurable to produce a light in one or more specific wavelengths or frequencies. For instance, the illuminant 106 includes one or more discrete light emitting elements, such as LEDs, OLEDs, fluorescent, other commonly known or understood lighting sources. In one arrangement, illuminant 106 is a broad-band LED. In one or more implementations, the illuminant 106 includes a lens, filter, screen, enclosure, or other elements (not shown) that are utilized in combination with the light source of the illuminant 106 to direct a beam of illumination, at a given wavelengths, to the eye 103. In one or more implementations the illuminant 106 is a light or flash that is associated with, or integrated into, a mobile phone, smartphone, tablet computer or similar portable computing device. In one implementation, the illuminant 106 is operable or configurable by an internal processor or other control circuit.

Alternatively, the illuminant 106 is operable or configurable by a remote processor or control device having one or more linkages or connections to the illuminant 106. As shown in FIG. 1, the illuminant 106 are directly connected to, and controlled in response to, signals provided by an image capture device 122.

Continuing with FIG. 1, light reflected off upon the eye 103 is captured or measured by an image capture device 122. Here, the image capture device 122 can be a camera or image capture device. For example, the image capture device 122 is a CMOS (Complementary Metal Oxide Semiconductor), CCD (charge coupled device), imaging sensor, photodiode array, or other light sensing device or image capture device and any associated hardware, firmware and software necessary for the operation thereof.

In a particular implementation, the image capture device 122 is configured to generate an output signal upon light being striking the image capture device 122 or a light sensing portion thereof. By way of non-limiting example, the image capture device 122 is configured to output a signal in response to light that has been reflected off of the eye 103 striking a light sensor or other sensor element integral or associated with the image capture device 122. For instance, the image capture device 122 is configured to generate a digital or analog signal that corresponds to the wavelength or wavelengths of light that a light sensor integral to the image capture device 122 after being reflected off of the eye 103. In one or more configurations, the image capture device 122 is configured to output spectral information, RGB information, or another form of multi-wavelength data representative of light reflected off the eye 103.

In one or more implementations, the image capture device 122 is a camera component of a smartphone, tablet or other portable communication device. Alternatively, the image capture device 122 is a standalone color measurement device that is configured to output data to one or more remote processors or computers.

In one non-limiting implementation, the image capture device 122 is a camera or image recording device integrated into a smartphone, tablet, cell phone, or other portable computing apparatus. In a further embodiment, the image capture device 122 is an “off the shelf” digital camera or web-camera connected or in communication with one or more computing devices.

The image capture device 122, in accordance with one embodiment, is a stand-alone device capable of storing local data corresponding to measurements made of the eye 103 within an integrated or removable memory. In an alternative implementation, the image capture device 122 is configured to transmit one or more measurements to a remote storage device or processing platform, such as processor 124. In configurations calling for remote storage of image data, the image capture device 122 is equipped or configured with network interfaces or protocols usable to communicate over a network, such as the internet.

Alternatively, the image capture device 122 is connected to one or more computers or processors, such as processor 124, using standard interfaces such as USB, FIREWIRE, Wi-Fi, Bluetooth, and other wired or wireless communication technologies suitable for the transmission measurement data.

The output signal generated by the image capture device 122 is transmitted to one or more processor(s) 124 for evaluation as a function of one or more hardware or software modules. As used herein, the term “module” refers, generally, to one or more discrete components that contribute to the effectiveness of the presently described systems, methods and approaches. Modules can include software elements, including but not limited to functions, algorithms, classes and the like. In one arrangement, the software modules are stored as software in the memory (not shown) of the processor 124. Modules also include hardware elements substantially as described below. In one implementation, the processor 124 is located within the same device as the image capture device 122. For example, the image capture device 122 and processor 124 are both incorporated into a smartphone, tablet computer or other portable computing device. However, in another implementation, the processor 124 is remote or separate from the image capture device 122.

In one configuration, the processor 124 is configured through one or more software modules to generate, calculate, process, output or otherwise manipulate the output signal generated by the image capture device 122. The processor 124 is configured to execute a commercially available or custom operating system, e.g., MICROSOFT WINDOWS, APPLE OSX, UNIX or Linux based operating system in order to carry out instructions or code.

The processor 124 may include one or more memory storage devices (memories). The memory is a persistent or non-persistent storage device (such as an IC memory element) that is operative to store the operating system in addition to one or more software modules. In accordance with one or more embodiments, the memory comprises one or more volatile and non-volatile memories, such as Read Only Memory (“ROM”), Random Access Memory (“RAM”), Electrically Erasable Programmable Read-Only Memory (“EEPROM”), Phase Change Memory (“PCM”), Single In-line Memory (“SIMM”), Dual In-line Memory (“DIMM”) or other memory types. Such memories can be fixed or removable, as is known to those of ordinary skill in the art, such as through the use of removable media cards or modules. In one or more embodiments, the memory of the processor 124 provides for the storage of application program and data files. One or more memories provide program code that the processor 124 reads and executes upon receipt of a start, or initiation signal.

The computer memories may also comprise secondary computer memory, such as magnetic or optical disk drives or flash memory, that provide long term storage of data in a manner similar to a persistent memory device. In one or more embodiments, the memory of the processor 124 provides for storage of an application program and data files when needed.

The processor 124 is configured to store data either locally in one or more memory devices. Alternatively, the processor 124 is configured to store data, such as measurement data or processing results, in a local or remotely accessible database 108. The physical structure of the database 108 may be embodied as solid-state memory (e.g., ROM), hard disk drive systems, RAID, disk arrays, storage area networks (“SAN”), network attached storage (“NAS”) and/or any other suitable system for storing computer data. In addition, the database 108 may comprise caches, including database caches and/or web caches. Programmatically, the database 108 may comprise flat-file data store, a relational database, an object-oriented database, a hybrid relational-object database, a key-value data store such as HADOOP or MONGODB, in addition to other systems for the structure and retrieval of data that are well known to those of skill in the art. The database 108 includes the necessary hardware and software to enable the processor 124 to retrieve and store data within the database 108.

In one implementation, each element provided in FIG. 1 is configured to communicate with one another through one or more direct connections, such as through a common bus. Alternatively, each element is configured to communicate with the others through network connections or interfaces, such as a local area network LAN or data cable connection. In an alternative implementation, the image capture device 122, processor 124, and database 108 are each connected to a network, such as the internet, and are configured to communicate and exchange data using commonly known and understood communication protocols.

In a particular implementation, the processor 124 is a computer, workstation, thin client or portable computing device such as an Apple iPad/iPhone® or Android® device or other commercially available mobile electronic computing device configured to receive and output data to or from database 108 and or image capture device 122.

In one arrangement, the processor 124 communicates with a local or remote display device 110 to transmit, displaying or exchange data. In one arrangement, the display device 110 and processor 124 are incorporated into a single form factor, such as a mobile computing device that includes an integrated display device, such as a smartphone or tablet computer. For example, the processor 124 is configured to send and receive data and instructions to the display device 110 for access or use by the user. Display device 110 includes one or more display devices configured to display data obtained from the processor 124. Furthermore, the display device 110 is also configured to send instructions to the processor 124. For example, where the processor 124 and the display device 110 are wirelessly linked using a wireless protocol, instructions can be entered into the display device that are executed by the processor. The display device 110 includes one or more associated input devices and/or hardware (not shown) that allow a user to access information, and to send commands and/or instructions to the processor 124 and the image capture device 122. In one or more implementations, the display device 110 can include a screen, monitor, display, LED, LCD or OLED panel, augmented or virtual reality interface or an electronic ink-based display device.

In one implementation, the processor 124 provides the processed measurement values to one or more cloud-based processors, computer, or server 126. For instance, a server 126 is a commercially available remote computing device. For example, the server 126 may be a collection of computers, servers, processors, cloud-based computing elements, micro-computing elements, computer-on-chip(s), home entertainment consoles, media players, set-top boxes, prototyping devices or “hobby” computing elements that are configured to receive signals, data, information or files from the processor 124, either locally or remotely over a network connection.

Furthermore, the processor 124 and the computing elements of server 126 can comprise a single processor, multiple discrete processors, a multi-core processor, or other type of processor(s) known to those of skill in the art, depending on the particular embodiment. In a particular example, the processor 124 and computing elements of the server 126 executes software code on the hardware of a custom or commercially available cellphone, smartphone, notebook, workstation or desktop computer configured to receive data or measurements captured by the image capture device 122 either directly, or through a communication linkage.

In one or more implementations, the processor 124 is further configured to access various peripheral devices and network interfaces. For instance, the processor 124 is configured to communicate over the internet with one or more remote servers, computers, peripherals or other hardware using standard or custom communication protocols and settings (e.g., TCP/IP, etc.).

Those possessing an ordinary level of skill in the requisite art will appreciate that additional features, such as power supplies, power sources, power management circuitry, control interfaces, relays, adaptors, and/or other elements used to supply power and interconnect electronic components and control activations are appreciated and understood to be incorporated.

By way of broad overview, it is one object of the present invention to provide a system to measure IOP that can easily be used outside the health professional's office. The method of the present invention does not require a special tonometer device or a health professional to be present. For example, in one or more implementations, the systems, methods and computer implemented processes described herein provide an end-to-end approach for the self-measurement of intraocular pressure.

Turning now to FIG. 7, the computing device is configured by one or more modules to initiate one or software applications that configure the processor 124 and the image capture device 122 to obtain one or more images of the eye 103 of a subject. For example, an initiation module 801 configures one or more elements the processor 124 to start an application. In one or more implementations, the processor 124 is instructed to provide to the user or the subject with one or more instructions. For instance, the initiation module 801 is configured to display on the display device 110 instructions for aligning one or more cameras or imaging devices 122 with the eye 103 of the user or subject. For instance, the initiation module 801 instructs the user with visual or audio commands to direct the camera of the “mobile device” to the eye.

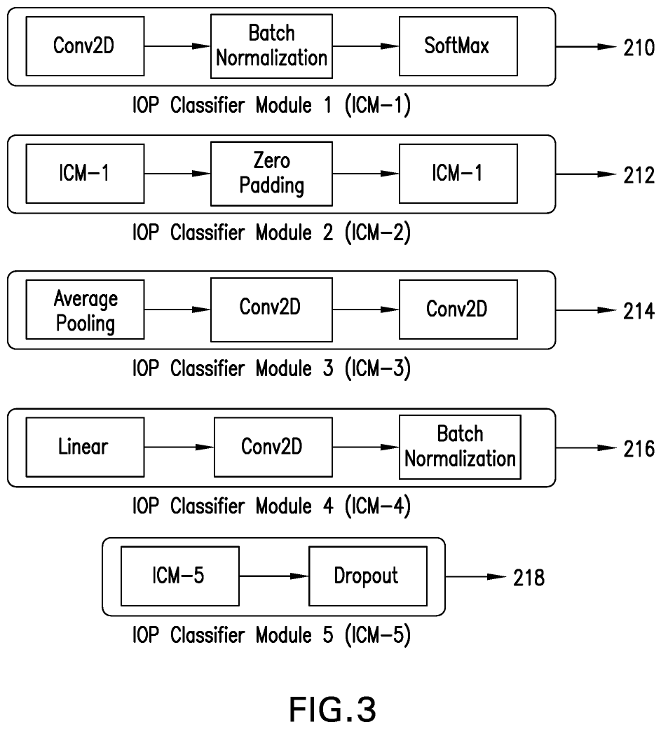

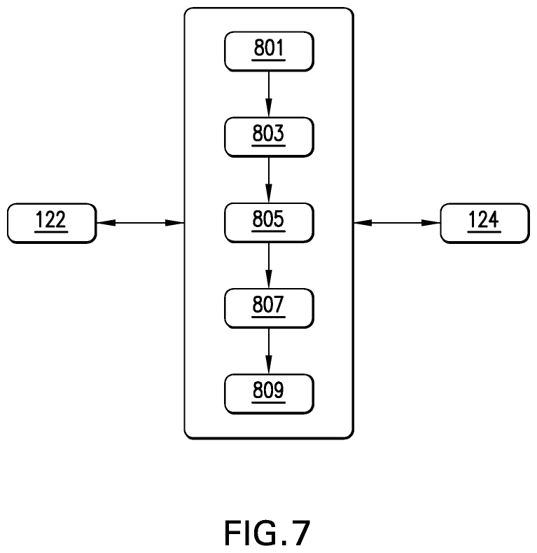

Once the eye has been aligned with the imagining device, the processor 124 is configured by one or more detecting modules 803 to evaluate the image captured of the eye 103 of the user. For example, the detecting module 803 configures the processor 124 to identify the pixels of an image or video stream of the eye 103 that correspond to the different areas of the eye 103. For example, based on the color of the image or live video stream, the processor 124 is configured by the detection module 803 to determine the location of the iris or pupil of the eye 103. Once the processor 124 is configured to identify the relevant portion of the eye, one or more submodules of the detection module 803 causes the processor to optimize camera zoom level, focus, light (such as the amount of light emitted by illuminant 106). In one or more implementations, the detection module 803 automatically adjusts the image capture device 122 so as to capture an image of the eye 103 that is in focus. By way of non-limiting example, as shown in FIG. 2, and FIG. 6, an eye detection submodule of the detection module 803 causes the processor 124 to evaluate the pixel coordinates of a bounding box that contains the eye. Here, the pixels encompassed by the bounding box are provided as an input to a camera adjustment module 102. The camera adjustment module 102, configured as a submodule of the detection module 803 configures the processor 124 to control the image capture device 122 so as to adjust at least zoom, focus and light parameters in order to focus the image capture device 122 on the given coordinates and take an image of the eye 103. For example, in one particular implementation, the image capture device 122 is obtain images of the eye 103 at resolution of at least 1600×1200 pixels.

Once the detection module 803 has configured the processor 124 to determine that the eye is within focus and properly lit, an image is obtained of the eye 103. For example, an image capture module 805 configures the processor 124 to capture an image of the eye 103 for further analysis and processing.

In one or more implementations, the image capture module 805 includes one or more submodules that configure the processor 124 to preprocess the captured image of the eye 103. For example, the image capture module 805 configures the processor 124 to implement de-noising, normalization, sharpening, edge detection, or other pre and post image processing routines and algorithms. By way of non-limiting example depicted in FIG. 1, once the picture of the eye is taken the image is transmitted to a server 126 for further pre-processing 128 and IOP classification 130.

In one particular implementation, upon receiving the image of the eye; the pre-processing submodule 128 of a processing module executing on the server 126 (such as, but not limited to image capture module 805) causes the image to be processed according to one or more further processing routines. For example, the pre-processing submodule 128 configures the server 126 to cause the image to undergo de-noise processing using a non-local means (NLM) image denoising algorithm. Once processed, the pre-processing module 128 configures one or more processors of the illuminant 106 to crop and resize the image to produce a 512×512 pixels image of the eye 103.

The processor 124 is configured by a neural network module 807. Here, the neural network module 807 configures the processor 124 or the server 126 to send the processed image or images of the eye 103 to a neural-network backed IOP classifier 130. In one or more implementations, the neural network is locally accessible by the processor 124. For example, the processor 124 is configured by a neural network module 807 to access a locally stored neural network configured to receive eye images and generate or output IOP data in response thereto. In one or more alternative arrangements, the server 126 is configured with the module 807, which configures a processor of the server 126 to apply the image of the eye 103 to the neural network. In a further implementation, the server 126 is configured to send the processed image to a further remote server, such as a cloud hosted server, for neural network processing. In this arrangement, the neural network module 807 configures the processor of the server 126 to transmit data through one or more wired or wireless network connections to the remote computer or server 126 hosting the neural network.

In a particular implementation, the neural network module 807 configures a processor to apply the images obtained by the image capture device 122 to an input layer of a neural network. By way of background on neural networks, neural networks are composed of an input layer, one or more intermediate layers, and an output layer. When a neural network is generated or initialized, the weights are randomly set to values near zero. At the start of a neural network training process, as would be expected, the untrained artificial neural network (ANN) does not perform the desired mapping of an input value to a target value very well. A training algorithm incorporating some optimization techniques must be applied to change the weights to provide an accurate mapping. The training is done in an iterative manner as prescribed by the training algorithm. Training data selection is generally a nontrivial task. An ANN is only as representative of the functional mapping as the data used to train it. Any features or characteristics of the mapping not included (or hinted at) within the training data will not be represented in the ANN.

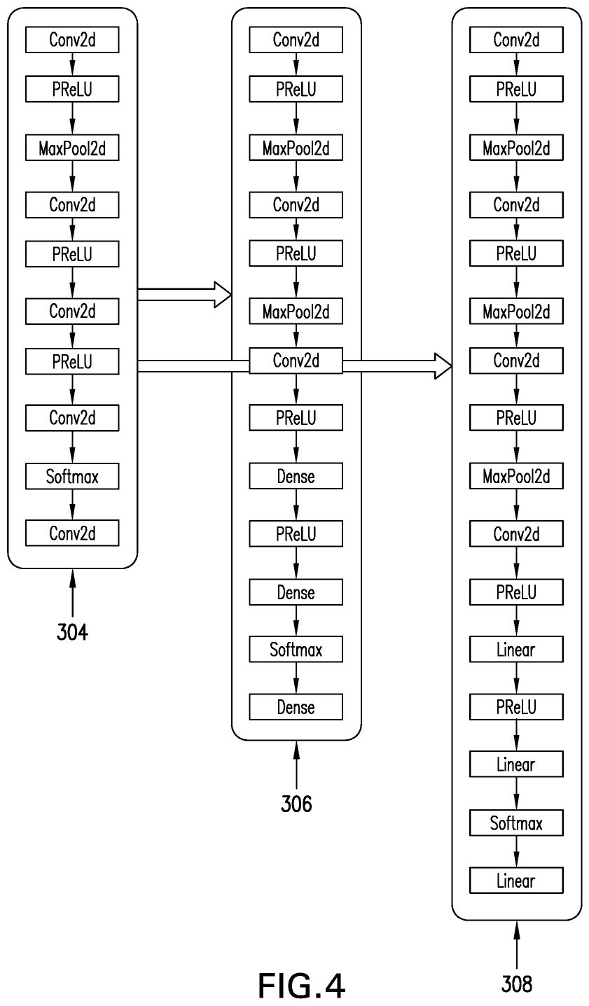

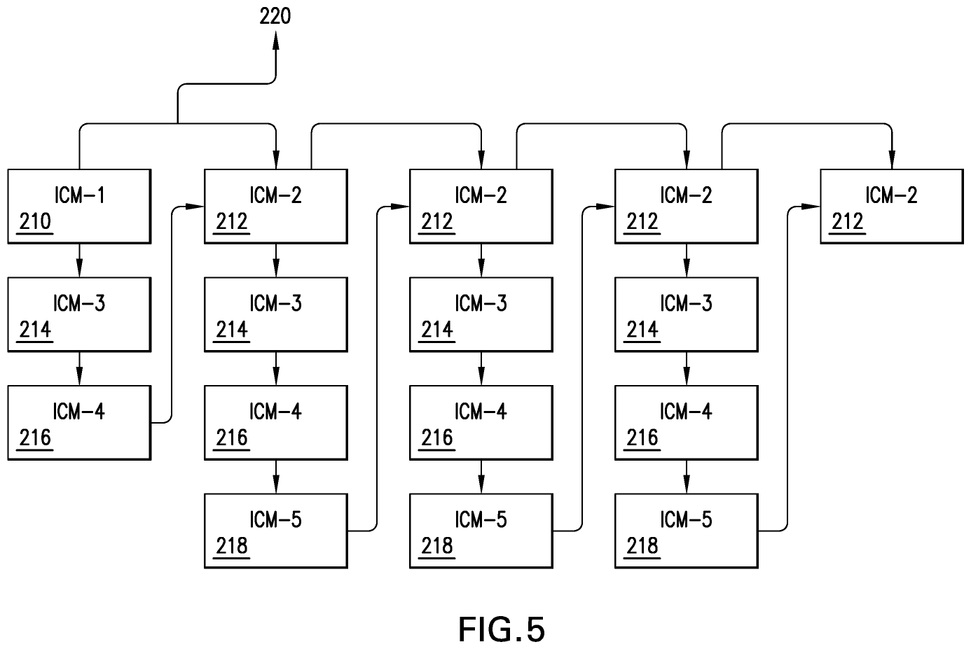

As provided in more detail herein, and depicted in FIG. 1, and FIGS. 3-5 the pre-processed eye image becomes an input to the IOP classifier 130. In one or more implementations, the processor of the server or processor 124 provides the image to the IOP classifier 130. For example, a processor of the illuminant 106 is configured by a classifier module 210 to utilize a convolutional neural networks and batch normalization to process the image of the eye to obtain a correlation between the eye image and a IOP measurement. In a further implementation a second classifier module 212 configures the processor of a server 126 to apply the image to a neural network having zero padding so as to prevent the data loss on the edges of the given eye image. In a further implementation, a third classifier module 214 utilizes average pooling to produce a summary statistic of its input and to reduce the spatial dimensions of the feature map to be used on the next layers. In one or more further implementations, a fourth classifier module 216 is configured to utilize a linear transformation together with convolutional neural networks to provide more learning capability to the overall model. Here, classifier module 216 also can be configured to utilize further batch normalization to accelerate learning process during training of the overall model. As shown with specific reference to FIG. 4, three different neural network architectures 304, 306, 308 can be employed by the server 126 executing a neural network module 807 are used to correctly identify the exact eye area.

By way of non-limiting further example, and returning to the process of eye detection, once the image capture device 122 is initiated and directed to the face of a user, the frames of the image capture device 122 become an input to the pre-processing of a neural network 304 configured for eye detection. The pre-processing resizes the frame images to 512×512 pixels and modifies the RGB image by first dividing the red, green and blue values by 255 then normalizes it by using mean (0.480, 0.460, 0.400) and standard deviation (0.240, 0.221, 0.232) values.

Here, the neural network 304 identifies the candidate rectangular pixel coordinates on the pre-processed face picture which potentially contains an eye. These rectangular pixel coordinates together with the image are inputs to the neural networks 306 and 308 which calculates the probability of including a real eye in that box. The outputs of the neural networks 306 and 308 are ensembled to a final result which are the coordinates of the box that contains the eye.

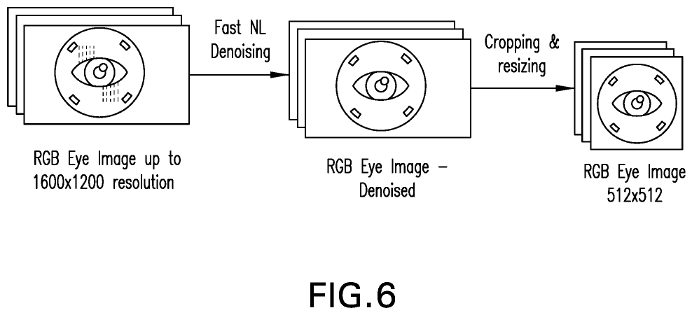

As depicted in FIG. 5, in one embodiment, all classifier modules 210, 212, 214, 216 and 218 are used multiple times to form a complex neural network with multiple skip connections 220. Here, it will be appreciated to those skilled in the art that the use of skip connections 220 prevents losing valuable information from the image on early layers.

Here, in one or more implementations, eye images are classified or correlated to given IOP determinations or evaluations. Using a training set of such labeled data, the neural network is configured to evaluate the and correlate the data within a given image with a statistically correlated IOP measurement. Thus, the neural network module 807 is configured to access such a suitably trained neural network (or ensemble of neural networks) such that one or more statistically correlated values for IOP are generated in response to an image of the eye 103.

In a further implementation, the statistical values correlated to the IOP measurement and other statistical data generated by the neural network are provided to the user. For example, a reporting module 809 provides information to the processor 124 that enables the processor 124 to generate a report with the results of the evaluation of the eye 103 of the user. In one or more implementation, the processor 124 receives data generated by a server configured by a reporting module 809. For example, the 124 receives data, file or other information that can be stored locally and used to generate a report 132 to the user of the IOP measured. Alternatively, the results are generated into a report by the reporting module 809 and are transmitted to the processor 124 for display to the user.

The following implementations present non-limiting approaches to carrying out the aims of the inventions described herein. For example, in one or more implementations, a mobile system backed by neural networks to detect eyes on a camera view frame includes: a “mobile device” with a camera and display component; a computing system installed in a “mobile device” in order to: run a convolutional neural network based model for identifying potential eye areas; run two convolutional neural network based model for calculating the probability of the existence of an eye in a given image. In a further example, of the exemplary system, the computing system is further configured to control the camera of a “mobile device” to focus on the detected eye. In a further example, of the exemplary system, the computing system is further configured to take a picture of the detected eye which can be used for IOP measurement. In yet a further example of the exemplary system, the computing system is further configured to send the picture of the eye to a server.

In one or more particular implementations, a computing system is provided that includes: one or more hardware computer processors and one or more storage; devices configured to store software instructions for executing by the one or more hardware computer processors in order to cause the computing system to: receive the eye image produced by the computing system in order to remove the noise from an eye picture produced by the computing system described herein and to crop and re-size an eye picture produced by the computing system.

In a particular implementation, a computer system is provided where the computing system is further configured to run a neural network for classifying the IOP value of a patient as normal or high wherein convolutional neural networks are used. In a further particular implementation of the computer systems described herein, the computing system is further configured to run a neural network for classifying the IOP value of a patient as normal or high wherein batch normalization techniques are used.

In a further implementation, the computing system is further configured to run a neural network for classifying the IOP value of a patient as normal or high wherein pooling techniques are used. In yet a further particular implementation, the computing system is further configured to generate a report that contains the classified IOP measurement results.

In an alternative configuration, the computing system is further configured to run a neural network for measuring the IOP value of a patient as a numerical value wherein convolutional neural networks are used. Furthermore, a computing system is further configured to run a neural network for measuring the IOP value of a patient as a numerical value wherein batch normalization techniques are used. In one or more alternative or further implementations, the computing system described herein is further configured to run a neural network for measuring the IOP value of a patient as a numerical value wherein pooling techniques are used.

In one or more implementations, a computer system is provided that is configured to generate a report that contains the measured IOP measurement results. In a further implementation, a computer system is configured to send the generated IOP report back to a user device or computing system described herein.

In one or more implementations, a computer system is provided that is configured to receive the IOP report generated from one or more remote processors or computers.

In one or more implementations, a computer system is provided wherein the computing system is further configured to display the IOP report received from a remote computer or processor to a user.

In a further implementation, a system for measurement of intraocular pressure (IOP) in at least one eye of a subject is provided where the system includes a handheld device; at least one source of light configure to illuminate the anterior aspect of the eye; at least one camera sensor configured to capture the light from the anterior aspect of the eye; an optical system mounted in the frame and configured to convey and focus the reflected and refracted light to the camera sensor; at least one memory storing instruction which is executed by at least one data processor; and at least one data processor.

In one or more implementations, a computer system is provided that includes a virtual reality, an augmented reality or a mixed reality engine with eye-tracking capabilities to collect information of the anterior aspect of the eye. In one further implementation, the computer system includes infrared sensors. In one or more further implementations, at least one eye-tracking system is configured to acquire images and videos of the anterior aspect of the eye. In one or more further implementations, the computer system is configured to provide a set of n images, wherein n is greater than or equal to 2. In a further implementation of the system described herein, the images include but are not limited to the lids, cornea, conjunctiva, anterior chamber, iridocorneal angle, iris, pupil and crystalline lens. In one or more further implementations, the computer system described herein uses at least one type of neural network (NN) or one type of support vector machine (SVM) to measure the IOP. For example, the machine learning algorithms provided include NN and the SVM that have been previously trained. Such training, in one or more implementations includes creating a training set of images from images of eyes with different levels of IOP selecting a tentative architecture for a NN to classify the level of IOP in the training set of images through an iterative process; a training database, wherein the training database includes, for each member of a training population comprised of users of a IOP test, an assessment dataset that includes at least data relating to a respective level of IOP an IOP score of the respective member; a training system including an expert system module configured to determine correlations between the respective user IOP test and the IOP for each member of the training population; a user testing platform configured to provide a user with an IOP test and receive user input regarding responses to the current IOP test; an analysis system communicatively coupled to the training system and the user testing platform, the computer system adapted to receive the user IOP generated in response to the current IOP test and to assign a IOP score for the user testing platform user using the correlations obtained from the training system; building an intermediate NN using the a set of images in different eye position; building an intermediate NN using the a set of images to identify different structures of the anterior aspect of the eye including but not limited to the lids, cornea, conjunctiva, anterior chamber, iridocorneal angle, iris, pupil and crystalline lens.

In a further particular implementation, the predetermined number of intermediate NNs is 23. In yet a further example, a system is provided that is configured to determine whether the NN meets the validation threshold comprises determining whether the NN has an error rate that is less than 15% on the validation set.

In yet a further example of the computer system described herein, a system for measurement the depth of the anterior chamber of the eye in at least one eye of a subject is provided, the system comprising, a handheld device; at least one source of light configure to illuminate the anterior aspect of the eye; at least one camera sensor configured to capture the light from the anterior aspect of the eye; an optical system mounted in the frame and configured to convey and focus the reflected and refracted light to the camera sensor; at least one memory storing instruction which is executed by at least one data processor; and at least one data processor.

The system described herein can be configured to include or comprise a virtual reality, an augmented reality or a mixed reality engine with eye-tracking capabilities to collect information of the anterior aspect of the eye. Such a computer system described can, in one or more implementations, include one or more infrared sensors. Likewise, such a computer system can include one or more eye-tracking system is configured to acquire images and videos of the anterior aspect of the eye.

In one or more further implementations, a computer system is provided that further provides a set of n images, wherein n is greater than or equal to 2. For example, the provided images include but are not limited to the lids, cornea, conjunctiva, anterior chamber, iridocorneal angle, iris, pupil and crystalline lens.

In one or more implementations, the computer system utilizes at least one type of neural network (NN) or one type of support vector machine (SVM) to measure the depth of the anterior chamber of the eye. In a further particular implementation, the NN and the SVM are previously trained by: creating a training set of images from images of eyes with different depth of the anterior chamber of the eye; selecting a tentative architecture for a NN to classify the depth of the anterior chamber of the eye in the training set of images through an iterative process; a training database, wherein the training database includes, for each member of a training population comprised of users of an anterior chamber depth test (gonioscopy), an assessment dataset that includes at least data relating to a respective level of depth of the anterior chamber of the eye; a depth of the anterior chamber of the eye score of the respective member; a training system including an expert system module configured to determine correlations between the respective user depth of the anterior chamber of the eye test and the depth of the anterior chamber of the eye for each member of the training population; a user testing platform configured to provide a user with a depth of the anterior chamber of the eye test and receive user input regarding responses to the current depth of the anterior chamber of the eye test; an analysis system communicatively coupled to the training system and the user testing platform, the computer system adapted to receive the user depth of the anterior chamber of the eye generated in response to the current depth of the anterior chamber of the eye test and to assign a depth of the anterior chamber of the eye score for the user testing platform user using the correlations obtained from the training system; building an intermediate NN using the a set of images in different eye position; and building an intermediate NN using the a set of images to identify different structures of the anterior aspect of the eye including but not limited to the lids, cornea, conjunctiva, anterior chamber, iridocorneal angle, iris, pupil and crystalline lens. By way of further example, the computer system described herein includes a predetermined number of intermediate NNs, such as 1, 2, 3 or 4.

In yet a further implementation, the computer system provided herein includes one or more NN that meets the validation threshold comprises determining whether the NN has an error rate that is less than 15% on the validation set.

In one or more further implementations of the approaches provided herein, a computer system is provided where the computing system is further configured to obtain an image of the eye, or a portion thereof, and evaluate the image according to one or more diagnostic or evaluation machine learning models or modules. For instance, a computer is configured to provide an image captured by an image capture device to one or more neural networks that have been trained to classify or determine the depth of the anterior chamber of the eye of a subject. By way of non-limiting example, such a trained neural network is configured to classify or provide an estimated value for the depth of the anterior chamber of the subject. In one or more further configurations, the neural network is configured to output a classification value (such as normal or abnormal) based on the evaluation of the image of the eye. In an alternative configuration, the neural network is configured to generate one or more evaluations of a predicted depth of the anterior chamber in units (such as but not limited to millimeters). In a further particular implementation of the computer systems described herein, the computing system is further configured to run a neural network for classifying the depth of the anterior chamber of a patient as normal or high wherein batch normalization techniques are used.

In one or more further implementations of the approaches provided herein, a computer system is provided where the computing system is further configured to obtain an image of the eye, or a portion thereof, and evaluate the image according to one or more diagnostic or evaluation machine learning models or modules. For instance, a computer is configured to provide an image captured by an image capture device to one or more neural networks that have been trained to classify or determine whether the patient is suffering from hypopyon. By way of non-limiting example, such a trained neural network is configured to classify or provide an estimated value for the amount of inflammation of various eye structures. In one or more further configurations, the neural network is configured to output a classification value (such as hypopyon or non-hypopyon) based on the evaluation of the image of the eye. In an alternative configuration, the neural network is configured to generate one or more evaluations that the subject is suffering from hypopyon. For example, the neural networks are configured to output a value that corresponds to a degree or percentage of certainty that the patient is suffering from hypopyon. In a further particular implementation of the computer systems described herein, the computing system is further configured to run a neural network for classifying the patient as suffering from one or more symptoms or conditions that correlate to hypopyon.

In one or more further implementations of the approaches provided herein, a computer system is provided where the computing system is further configured to obtain an image of the eye, or a portion thereof, and evaluate the image according to one or more hyphema diagnostic or evaluation machine learning models or modules. For instance, a computer is configured to provide an image captured by an image capture device to one or more neural networks that have been trained to classify or determine whether the patient is suffering from hyphema. By way of non-limiting example, such a trained neural network is configured to classify or provide an estimated value for the amount of blood present in the anterior chamber of the eye. In one or more further configurations, the neural network is configured to output a classification value (such as hyphema or non-hyphema) based on the evaluation of the image of the eye. In an alternative configuration, the neural network is configured to generate one or more evaluations that the subject is suffering from hyphema. For example, the neural networks are configured to output a value that corresponds to a degree or percentage of certainty that the patient is suffering from hyphema. In a further particular implementation of the computer systems described herein, the computing system is further configured to run a neural network for classifying the patient as suffering from one or more symptoms or conditions that correlate to hyphema.

In one or more further implementations of the approaches provided herein, a computer system is provided where the computing system is further configured to obtain an image of the eye, or a portion thereof, and evaluate the image according to one or more inflammation diagnostic or evaluation machine learning models or modules. For instance, a computer is configured to provide an image captured by an image capture device to one or more neural networks that have been trained to classify or determine cellular or other inflammation of the eye of a subject. By way of non-limiting example, such a trained neural network is configured to classify or provide an estimated value for the amount of inflammation of the eye. In one or more further configurations, the neural network is configured to output a classification value (such as normal or abnormal) based on the evaluation of the image of the eye. In an alternative configuration, the neural network is configured to generate one or more evaluations of a predicted amount of inflammation found within one or more intraocular structures, such as percentage of structures that are inflamed. In a further particular implementation of the computer systems described herein, the computing system is further configured to run a neural network for classifying the inflammation of one or more intraocular structures as correlated to a specific ailment or condition of a patient. For instance, the neural network is configured to identify the amount of inflammation detected as normal or high wherein batch normalization techniques are used.

In one or more further implementations of the approaches provided herein, a computer system is provided where the computing system is further configured to obtain an image of the eye, or a portion thereof, and evaluate the image according to one or more iridocorneal angle diagnostic or evaluation machine learning models or modules. For instance, a computer is configured to provide an image captured by an image capture device to one or more neural networks that have been trained to classify or determine the aperture of the iridocorneal angle of the eye of a subject. By way of non-limiting example, such a trained neural network is configured to classify or provide an estimated value for the aperture size of the iridocorneal angle of the subject. In one or more further configurations, the neural network is configured to output a classification value (such as normal or abnormal) based on the evaluation of the image of the eye. In an alternative configuration, the neural network is configured to generate one or more evaluations of a predicted iridocorneal angle aperture size in units (such as but not limited to millimeters). In a further particular implementation of the computer systems described herein, the computing system is further configured to run a neural network for classifying the aperture size of the iridocorneal angle of a patient as normal or high wherein batch normalization techniques are used.

In one or more further implementations of the approaches provided herein, a computer system is provided where the computing system is further configured to obtain an image of the eye, or a portion thereof, and evaluate the image according to one or more central cornea diagnostic or evaluation machine learning models or modules. For instance, a computer is configured to provide an image captured by an image capture device to one or more neural networks that have been trained to classify or determine the thickness of the central corneal area of the eye of a subject. By way of non-limiting example, such a trained neural network is configured to classify or provide an estimated value for the thickness of the central cornea of the subject. In one or more further configurations, the neural network is configured to output a classification value (such as normal or abnormal thickness) based on the evaluation of the image of the eye. In an alternative configuration, the neural network is configured to generate one or more evaluations of a predicted thickness of the central cornea in units (such as but not limited to millimeters). In a further particular implementation of the computer systems described herein, the computing system is further configured to run a neural network for classifying the patient has having one or more conditions based on a predicted or evaluated thickness of the central cornea of a patient wherein batch normalization techniques are used.

In one or more further implementations of the approaches provided herein, a computer system is provided where the computing system is further configured to obtain an image of the eye, or a portion thereof, and evaluate the image according to one or more topological diagnostic or evaluation machine learning models or modules. For instance, a computer is configured to provide an image captured by an image capture device to one or more neural networks that have been trained to classify or determine the topology of one or more intraocular structures of the eye of a subject. By way of non-limiting example, such a trained neural network is configured to classify or provide an analysis of the topology of one or more intraocular structures chamber of the subject. In one or more further configurations, the neural network is configured to output a classification value (such as normal or abnormal topologies) based on the evaluation of one or more intraocular structures contained within the image of the eye. In an alternative configuration, the neural network is configured to generate one or more evaluations of a predicted ailment or condition based on the topology of one or more intraocular structures identified by the machine learning models.

While this specification contains many specific embodiment details, these should not be construed as limitations on the scope of any embodiment or of what can be claimed, but rather as descriptions of features that can be specific to particular embodiments. Certain features that are described in this specification in the context of separate embodiments can also be implemented in combination in a single embodiment. Conversely, various features that are described in the context of a single embodiment can also be implemented in multiple embodiments separately or in any suitable sub-combination. Moreover, although features can be described above as acting in certain combinations and even initially claimed as such, one or more features from a claimed combination can in some cases be excised from the combination, and the claimed combination can be directed to a sub-combination or variation of a sub-combination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing can be advantageous. Moreover, the separation of various system components in the embodiments described above should not be understood as requiring such separation in all embodiments, and it should be understood that the described program components and systems can generally be integrated together in a single software product or packaged into multiple software products.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises” and/or “comprising”, when used in this specification, specify the presence of stated features, integers, steps, operations, elements, and/or components, but do not preclude the presence or addition of one or more other features, integers, steps, operations, elements, components, and/or groups thereof.

It should be noted that use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having the same name (but for use of the ordinal term) to distinguish the claim elements. Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

Particular embodiments of the subject matter described in this specification have been described. Other embodiments are within the scope of the following claims. For example, the actions recited in the claims can be performed in a different order and still achieve desirable results. As one example, the processes depicted in the accompanying figures do not necessarily require the particular order shown, or sequential order, to achieve desirable results. In certain embodiments, multitasking and parallel processing can be advantageous.

Publications and references to known registered marks representing various systems cited throughout this application are incorporated by reference herein. Citation of any above publications or documents is not intended as an admission that any of the foregoing is pertinent prior art, nor does it constitute any admission as to the contents or date of these publications or documents. All references cited herein are incorporated by reference to the same extent as if each individual publication and references were specifically and individually indicated to be incorporated by reference.

While the invention has been particularly shown and described with reference to a preferred embodiment thereof, it will be understood by those skilled in the art that various changes in form and details may be made therein without departing from the spirit and scope of the invention. As such, the invention is not defined by the discussion that appears above, but rather is defined by the claims that follow, the respective features recited in those claims, and by equivalents of such features.

What is claimed is:

1. A system for measurement of an intraocular pressure (IOP) of at least one eye of a subject comprising,
a handheld device;
at least one source of light configured to illuminate an anterior aspect of the eye and produce a reflected and refracted light from the anterior aspect of the eye;
at least one camera sensor configured to capture the reflected and refracted light from the anterior aspect of the eye;
an optical system mounted in the frame of the device and configured to convey and focus the reflected and refracted light to the at least one camera sensor;
at least one data processor; and
at least one memory storage device that stores instruction which is executed by the at least one data processor.

2. The system of claim 1, further comprising an engine selected from the group consisting of a virtual reality engine, an augmented reality engine and a mixed reality engine, the engine including eye-tracking capabilities to collect information from the anterior aspect of the eye.

3. The system of claim 1, further comprising infrared sensors.

4. The system of claim 1, wherein at least one eye-tracking system is configured to acquire images and videos of the anterior aspect of the eye.

5. The system of claim 4, that further provides a set of n images, wherein n is greater than or equal to 2.

6. The system of claim 4, wherein the images are images selected from the group consisting of images of the lids, images of the cornea, images of the conjunctiva, images of the anterior chamber, images of the iridocorneal angle, images of the iris, images of the pupil, images of the crystalline lens, and combinations thereof.

7. The system of claim 1, wherein the system uses at least one type of neural network (NN) or one type of support vector machine (SVM) to measure the IOP.

8. The system of claim 7, wherein the NN and the SVM are previously trained by a training system, the training comprising:
creating a training set of images from images of eyes for each member of a training population, the images of eyes including different levels of IOP;
selecting a tentative architecture for a NN to classify the level of IOP in the training set of images through an iterative process;
using a training database,
wherein the training database includes, for each member of the training population each with an associated IOP test, an assessment dataset that includes at least data relating to the level of IOP for each member of the training population,
wherein the training population includes a test member with an associated IOP test;
assigning an IOP score of the test member of the training population;
configuring an expert system module of a training system including to determine correlations between the level of IOP of the test member and the IOP for each member of the training population;

9. The system of claim 7, further comprising:
a user testing platform configured to provide the subject with an IOP test and receive user input regarding responses to the IOP test;
an analysis system communicatively coupled to the training system and the user testing platform, the analysis system adapted to receive an IOP for the subject generated in response to the a subject IOP test and to assign a IOP score for the subject using the correlations obtained from the training system;
one or more intermediate NN built using a set of images of the subject in different eye positions; and
one or more intermediate NN built using a set of images of the subject to identify different structures of the anterior aspect of the eye including but not limited to images of the lids, images of the cornea, images of the conjunctiva, images of the anterior chamber, images of the iridocorneal angle, images of the iris, images of the pupil and images of the crystalline lens;

10. The system of claim 9 wherein the number of intermediate NNs is 23.

11. The system of claim 7, wherein the NN is assigned an error rate relative to a validation set, wherein an error rate of less than 15% identifies the NN as having passed a validation threshold.

12. The system of claim 1, wherein the handheld device is a mobile device and the system is further configured to control the camera of the mobile device to focus on the at least one eye of the subject. 13. The system of claim 1, wherein the system is further configured to take an eye picture of the at least one eye of the subject which can be used for IOP measurement.

13. The system of claim 1, wherein the system is further configured to send the eye picture of the eye to a server.

14. The system of claim 1, wherein the system is further configured to receive the eye picture, to remove the noise from the eye picture, and to crop and re-size the eye picture.

15. The computing system of claim 1, wherein the system is further configured to run a neural network for measuring the IOP value of a patient as a numerical value wherein convolutional neural networks are used.

16. The computing system of claim 14, wherein the system is further configured to run a neural network for measuring the IOP value of a patient as a numerical value wherein batch normalization techniques are used.

17. The computing system of claim 14, wherein the system is further configured to run a neural network for measuring the IOP value of a patient as a numerical value wherein pooling techniques are used.

18. The computing system of claim 1, wherein the system is further configured to generate a report that contains classified IOP measurement results.

19. The computing system of claim 1, wherein the system is further configured to run a neural network for measuring the IOP value of a patient as a numerical value wherein convolutional neural networks are used.

20. The computing system of claim 1, wherein the system is further configured to transmit and display the IOP report received from the computing system of claim 1 to the subject.
